(19) United States
(12) Plant Patent Application Publication
(10) Pub. No.: US 2014/0283251 P1
(43) Pub. Date: Sep. 18, 2014

(54) Hydrangea macrophylla plant named 'show stopper'

(50) ;

(71) Applicant: Joshua H. Kardos, Watkinsville, GA (US)

(72) Inventor: Joshua H. Kardos, Watkinsville, GA (US)

(73) Assignees: University of Georgia Research Foundation, Inc., Athens, GA (US); Plant Introductions, Inc., Watkinsville, GA (US)

(21) Appl. No.: 13/998,265

(22) Filed: Oct. 17, 2013

Related U.S. Application Data
(60) Provisional application No. 61/784,974, filed on Mar. 14, 2013.

(51) Int. Cl. A01H 5/00

(52) U.S. Cl. PLT/250

(57) ABSTRACT

A new and distinct cultivar of Hydrangea macrophylla plant named ‘Show Stopper’, characterized by its remontant (reblooming) trait, flowering on old wood and new growth of the season; dark pink mophead inflorescences or dark blue inflorescences in non-aluminum or aluminum based media, respectively; compact, mounded growth habit; lustrous, dark green foliage; strong stems that hold the inflorescences upright; and moderate resistance to powdery mildew.

### CROSS REFERENCE TO RELATED APPLICATION

This application claims the benefit of U.S. Provisional Application No. 61/784,974 filed Mar. 14, 2013, the disclosure of which is incorporated by reference in its entirety.

### GENUS AND SPECIES OF PLANT CLAIMED

Hydrangea macrophylla (Thunb.) Ser.

### VARIETY DENOMINATION

‘Show Stopper’

### BACKGROUND OF THE INVENTION

The present invention relates to a new and distinct cultivar of Hydrangea macrophylla, a member of the Hydrangeaceae family, hereinafter referred to by its cultivar name ‘Show Stopper’. This cultivar is grown primarily as an ornamental for landscape use and for use as a potted plant.

The cultivar originated from a controlled cross-pollination of Hydrangea macrophylla ‘Penny Mac’ (unpatented) crossed with Hydrangea macrophylla ‘Mathilda Gutges’ (unpatented) in Athens, Ga. in 2005, and was selected from the progeny of this cross by continued evaluation for growth habit, improved leaf and flower characteristics, and increased resistance to powdery mildew. The cultivar ‘Show Stopper’ originated and was discovered in a cultivated environment in Athens, Ga.

‘Show Stopper’ has been asexually reproduced by softwood cuttings since 2006 in Athens, Ga. and in Watkinsville, Ga. The characteristics of the cultivar have been stable and reproduced true to type in successive vegetative generations.

### SUMMARY OF THE INVENTION

‘Show Stopper’ has not been observed under all possible environmental conditions. The phenotype may vary somewhat with changes in light, temperature, soil and rainfall without, however, any variance in genotype.

The following traits have been observed and represent the characteristics of the new cultivar. In combination these characteristics distinguish ‘Show Stopper’ from all other varieties in commerce known to the inventor. 1) Remontant (reblooming) trait, flowering on old wood and new growth of the season. 2) Dark pink mophead inflorescences or dark blue inflorescences in non-aluminum or aluminum based media, respectively. 3) Compact, mounded growth habit. 4) Lustrous, dark green foliage. 5) Strong stems that hold the inflorescences upright. 6) Moderate resistance to powdery mildew.

‘Show Stopper’ is distinguished from its female parent ‘Penny Mac’ by its growth habit and flower and foliage color. ‘Show Stopper’ has a compact, mounded growth habit, dark pink or dark blue inflorescences, and lustrous, dark green foliage, whereas ‘Penny Mac’ has an overall larger, rounded growth habit, lighter pink or blue inflorescences, and matte, lighter green foliage. ‘Show Stopper’ is distinguished from its male parent ‘Mathilda Gutges’ by its growth habit, flower color, and reblooming trait. ‘Show Stopper’ has a compact, mounded growth habit, dark pink or dark blue inflorescences, and is a rebloomer, whereas ‘Mathilda Gutges’ has an overall larger, rounded growth habit, rich pink or violet-blue inflorescences, and is not a rebloomer. There are no other cultivars of Hydrangea macrophylla with this combination of traits known to the inventor.

‘Show Stopper’ can be compared to the cultivar ‘Bountiful Bouquets’ (Plant Patent Applied For) but differs in the following characteristics. ‘Show Stopper’ has a smaller, compact, mounded growth habit, whereas ‘Bountiful Bouquets’ has a larger, compact, rounded growth habit. ‘Show Stopper’ has dark pink or dark blue inflorescences, whereas ‘Bountiful Bouquets’ has medium pink or medium blue inflorescences. ‘Show Stopper’ has lustrous, dark green foliage, whereas ‘Bountiful Bouquets’ has matte, dark green foliage.

### BRIEF DESCRIPTION OF THE DRAWINGS

The accompanying illustrations show characteristics of the new cultivar in photographs as true to color as is reasonably possible to make in illustrations of this nature. Colors in the photographs may differ slightly from the color values cited in the detailed botanical description which accurately describe the colors of the new Hydrangea.

FIG. 1 illustrates a ‘Show Stopper’ plant grown in the ground in the presence of aluminum.

FIG. 2 illustrates an inflorescence on ‘Show Stopper’ grown in the presence of aluminum.

FIG. 3 illustrates an inflorescence on ‘Show Stopper’ grown in the absence of aluminum.

### DETAILED DESCRIPTION

In the following description, color references are made to The Royal Horticultural Society Colour Chart, 2007 Edition, except where general terms of ordinary dictionary significance are used. Plants used for the description were grown in 11.8 L containers in a medium consisting of composted pine bark that was amended with 45 grams of aluminum sulphate and a pH of 5.7. Plants were grown under 50% shade under outdoor conditions in a nursery in Watkinsville, Ga. Plants were about 2 years old when the description was recorded.
Botanical classification: ‘Show Stopper’ is a cultivar of Hydrangea macrophylla. Parentage: The current variety is a progeny from a controlled cross-pollination of Hydrangea macrophylla ‘Penny Mac’ crossed with Hydrangea macrophylla ‘Mathilda Gutges’. Propagation: Vegetatively by stem cuttings.
Plant description: The claimed variety is a compact, mounded deciduous shrub. The original plant, now about 8-years-old in the ground, is about 97 cm in height from the soil level to the top of the inflorescences, and about 175 cm in diameter. Freely branching. The plant is cold hardy in USDA Zones 5 to 9.
Stems: First year stems have a diameter of about 7 mm, a round shape, and a stout, glabrous and lustrous texture with very fine pubescence. Exfoliation: on second year and older stems, flaky and stringy. Internodes have a length of about 6.7 cm. The stem color is 144B. Second year stems have a diameter of about 8 mm or more and are N199D in color.
Vegetative buds: Arrangement: opposite; Shape: ovoid; Size: about 4 mm in length, about 3 mm in width; Color: 144B at the base, gradually changing to 176A at the apex.
Leaves:
Size: about 18.1 cm in length and about 10.3 cm in width. Shape: ovate, with acuminate apex, cuneate base and serrate margin. Texture: thick and leathery. Color: emerging leaves are N137B on the upper and lower surfaces; mature leaves on the upper surface are 147A, and 147B on the lower surface.
Venation: pinnate, veins are 144C on the upper surface and 144D on the lower surfaces.
Petiole: about 3.8 cm in length and about 4.5 mm in diameter, glabrous texture, and 144C in color.
Flowers:
Inflorescence bloom period: early summer to first frost in fall. An inflorescence contains about 131 individual fertile flowers. An inflorescence contains about 429 individual sterile florets. Inflorescence shape and size: mophead, about 9.8 cm in height and about 19.5 cm in diameter. The inflorescence is effective for about 8 weeks. The peduncle is about 6.9 cm in length, is finely pubescent, and is 144B in color. The pedicels are about 2.4 cm in length, finely pubescent, and 84C in color.
Flower bud size: about 3 mm in length, about 3 mm in diameter. Flower bud shape: round; Color: 91B. Sterile florets are about 3.7 cm in diameter. Each floret contains 4 sepals about 1.8 cm in length and about 1.5 cm in width. Sterile floret shape: ovoid with obtuse apex, acute base, and entire margin; Texture: smooth with no pubescence; Color at maturity: the upper surface is 92A and the lower surface is 92B.
Petals (fertile flowers): Size: 5 petals per flower, about 4 mm in length and about 2 mm in width; Shape: ovate, with acuminate apex, truncate base, and entire margin; Texture: smooth with no pubescence; Color: at peak of bloom the upper surface of the petals is 97A and the lower surface is 95D.
Number of Stamens: 6 to 10. Anthers: about 1 mm in length and about 0.5 mm in width, 98D in color. Filaments: about 4 mm in length and NN155D in color. Pollen: NN155B in color, produced in moderate quantities. Pistil: superior, about 3 mm in length and about 1.5 mm in width, 96D in color. Stigma: 2 or 3 per pistil, round in shape and 96D in color. Style: about 2 mm in length and tubular in shape, 96B in color.
Fruit: The capsule fruit is ovoid, about 3 mm in length and about 2 mm in width. Color at maturity is 200C. The number of fruit per infructescence varies widely. Seed: The seeds are round, about 0.5 mm in length and about 0.5 mm in width, close to 199B, and each capsule contains about 50 seeds.
Disease/pest resistance: Moderately resistant to powdery mildew. No other pest or disease resistance/susceptibility has been observed. ‘Show Stopper’ is cold hardy from USDA Cold Hardiness Zones 5-9.

I claim:

1. A new and distinct Hydrangea macrophylla plant named ‘Show Stopper’, substantially as illustrated and described herein.
